(19) United States
(12) Patent Application Publication
(10) Pub. No.: US 2018/0296487 A1
(43) Pub. Date: Oct. 18, 2018

(54) SUSTAINED RELEASE INJECTABLE NEUROSTEROID FORMULATIONS

(71) Applicant: MARINUS PHARMACEUTICALS, INC., Radnor, PA (US)

(72) Inventors: Michael Saporito, West Chester, PA (US); Mingbao Zhang, Millwood, NY (US); Raymond C. Glowaky, Killingworth, CT (US); David Czekai, Haverford, PA (US)

(21) Appl. No.: 15/956,385

(22) Filed: Apr. 18, 2018

Related U.S. Application Data
(60) Provisional application No. 62/486,599, filed on Apr. 18, 2017.

(51) Int. Cl. A61K 9/14; A61K 9/00; A61K 31/57

(52) U.S. Cl. CPC A61K 9/146; A61K 31/57; A61K 9/145; A61K 9/0019

(57) ABSTRACT

The disclosure provides a sustained release injectable neurosteroid formulation comprising neurosteroid particles having a D50 of less than 10 microns, the neurosteroid particles comprising a neurosteroid of Formula I:

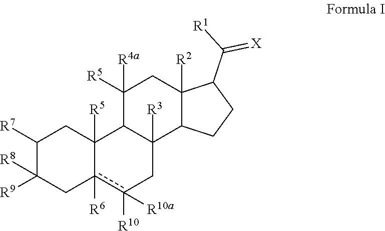

or a pharmaceutically acceptable salt thereof, wherein: [symbol] is a double or single bond and the variables, e.g., R¹, R², R³, R⁴, R^{4a}, R⁵, R⁶, R⁷, R⁸, R⁹, R¹⁰, and R^{10a} are described herein. The formulation comprises neurosteroid particles comprising the neurosteroid and a polymeric surface stabilizer and provides an effective plasma concentration of the neurosteroid at steady state for at least 48 hours, and in some embodiments for at least 4 weeks. The sustained release injectable neurosteroid formulation can formulated for intramuscular or subcutaneous administration. The disclosure provides a method of treating a patient having seizures, anxiety, agitation, depression (including post-partum depression), schizophrenia, post-traumatic stress disorder, or tremors by administering the sustained release neurosteroid formulation to the patient. In certain embodiments the neurosteroid is ganaxolone.

### CROSS REFERENCE TO RELATED APPLICATION

This application claims priority of U.S. Provisional application No. 62/486,599, filed Apr. 18, 2017, which is hereby incorporated by reference in its entirety.

### BACKGROUND

Pregnane neurosteroids are a class of compounds useful as anesthetics, sedatives, hypnotics, anxiolytics, and anticonvulsants. These compounds are marked by very low aqueous solubility, which limits their formulation options. Long-acting, sustained release injectable formulations of pregnane neurosteroids are particularly desirable as these compounds are useful for clinical indications that are best treated in an outpatient setting but for which stable compound blood levels are particularly desired. These disorders include seizure disorders, depression, including postpartum depression, and psychotic disorders.

There are no sustained release injectable neurosteroid formulations. Thus, there exists the need for sustained release injectable neurosteroid formulations for treating CNS disorders, including seizure disorders, anxiety, depression, and pain. This disclosure fulfills this need by providing sustained release injectable neurosteroid formulations and provides additional advantages that are described herein.

### SUMMARY

The disclosure provides a sustained release injectable neurosteroid formulation comprising neurosteroid particles having a D50 (volume weighted median diameter) of less than 10 microns and the neurosteroid particles comprising a pregnane neurosteroid, at least one surface stabilizer, for example a polymer surface stabilizer such as hydroxyethyl starch, dextran, or povidone, and in some embodiment an additional surface stabilizer, such as a surfactant. The disclosed sustained release formulations have a longer neurosteroid half-life in vivo than immediate release neurosteroid formulation. The sustained release formulations may also be considered long acting in that they provide and effective amount of neurosteroid in vivo for an extended time after administration. In preferred embodiments neurosteroid efficacy persists for at least 48 hours after injections and may persist for 4 weeks or longer after injection. An embodiment of the formulation comprises the neurosteroid particles in an aqueous suspension. The disclosure also provides a lyophilized powder of the pregnane neurosteroid nanoparticle formulation that may be reconstituted in an appropriate diluent, such as water, for injection.

The disclosure provides a sustained release injectable neurosteroid formulation comprising neurosteroid particles having a D50 of less than 10 microns, the neurosteroid particles comprising

a) a neurosteroid of Formula I:

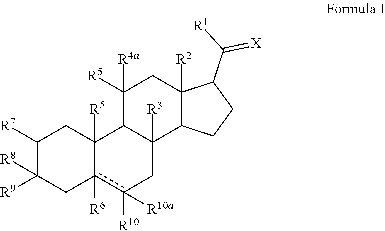

or a pharmaceutically acceptable salt thereof, wherein:

[symbol] is a double or single bond;

X is O, S, or NR¹¹;

R¹ is hydrogen, hydroxyl, optionally substituted alkyl, optionally substituted heteroalkyl, optionally substituted heteroaryl, optionally substituted heteroarylalkyl, optionally substituted aryl, or optionally substituted arylalkyl;

R⁴ is hydrogen, optionally substituted alkyl, optionally substituted cycloalkyl, or optionally substituted (cycloalkyl)alkyl, or —OR⁴⁰, where R⁴⁰ is hydrogen, optionally substituted alkyl, optionally substituted cycloalkyl, or optionally substituted (cycloalkyl)alkyl, or optionally substituted C₃-C₆carbocycle;

R^{4a} is hydrogen or R⁴ and R^{4a} are taken together to form an oxo (═O) group;

R², R³, R⁵, and R⁶, are each independently hydrogen, hydroxyl, halogen, optionally substituted alkyl, optionally substituted cycloalkyl, or optionally substituted (cycloalkyl)alkyl, or optionally substituted heteroalkyl;

R⁷ is hydrogen, halogen, optionally substituted alkyl, optionally substituted C₃-C₆carbocycle, optionally substituted (C₃-C₆carbocycle)alkyl or —OR⁷⁰ where R⁷⁰ is hydrogen, optionally substituted alkyl, optionally substituted C₃-C₆carbocycle, or optionally substituted (C₃-C₆carbocycle)alkyl;

R⁸ is hydrogen, optionally substituted alkyl or optionally substituted C₃-C₆carbocycle, and R⁹ is hydroxyl; or

R⁸ and R⁹ are taken together to form an oxo group;

R¹⁰ is hydrogen, halogen, hydroxyl, optionally substituted alkyl, optionally substituted heteroalkyl, optionally substituted C₃-C₆carbocycle, or optionally substituted (C₃-C₆carbocycle)alkyl, and R^{10a} is hydrogen, halogen, or optionally substituted alkyl, provided that if [symbol] is a double bond R^{10a} is absent;

each alkyl is a C₁-C₁₀alkyl and optionally contains one or more single bonds replaced by a double or triple bond;

each heteroalkyl group is an alkyl group in which one or more methyl group is replaced by an independently chosen —O—, —S—, —N(R¹¹)—, —S(═O)— or —S(═O)₂—, where R¹¹ is independently chosen at each occurrence and is hydrogen, alkyl, or alkyl in which one or more methylene group is replaced by —O—, —S—, —NH, or —N-alkyl; and

b) at least one polymeric surface stabilizer;

wherein the formulation provides an effective plasma concentration of the compound at steady state for at least 48 hours.

The disclosure also includes embodiments of the above injectable neurosteroid formulation in which formulation contains neurosteroid particles have a D50 of less than 10 microns, less than 5 microns, or 1 to 5 microns, which contain a surfactant as an additional surface stabilizer. The disclosure also includes neurosteroid particles having any of the D50 ranges listed above, the neurosteroid particles comprising

a) a compound or salt of Formula I;

b) a polymeric surface stabilizer; and

c) at least one additional surface stabilizer, wherein the additional surface stabilizer is a surfactant.

In certain embodiments the neurosteroid is ganaxolone (GNX) or allopregnanolone (ALLO). In certain embodiments the neurosteroid is ganaxolone.

The disclosure also provides a method of treating a patient having a seizure disorder, stroke, depression, including postpartum depression and treatment resistant depression in post-menopausal women, or traumatic brain injury, comprising administering an effective amount of the injectable neurosteroid nanoparticle formulation comprising a neurosteroid of Formula I. In certain embodiments the disclosure also provides a method for treating human patients in need of treatment for a central nervous system disorder selected from the group consisting of epileptic seizures, infantile spasms, essential tremor, anxiety, stress, panic, depression, postpartum depression, insomnia, premenstrual syndrome, post-traumatic stress disorder, substance abuse withdrawal, hypertension, pain, migraine headache, headache associated with pre- and peri-menstrual period, Neimann Pick disease Type-C and Mucolipidosis type IV lipid accumulation, mucolipidosis Type IV lipid accumulation, AIDS-related dementia, Alzheimer's disease, Huntington's disease, and Parkinson's disease comprising administering an effective amount of the injectable neurosteroid nanoparticle formulation comprising a neurosteroid of Formula I.

The disclosure includes methods of treatment in which the neurosteroid is the only active agent and methods in which the neurosteroid, of the neurosteroid nanoparticle formulation, is a first active agent and is administered in combination with an additional active agent.

The disclosure includes methods of treatment which include administration schedules for the neurosteroid nanoparticle formulation, in which the neurosteroid is the only active agent or in which the method includes treatment with at least one additional active agent.

### DETAILED DESCRIPTION

### Definitions

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely for illustration and does not pose a limitation on the scope of the invention unless otherwise claimed. No language in the specification should be construed as indicating any non-claimed element as essential to the practice of the invention.

The terms “a” and “an” do not denote a limitation of quantity, but rather denote the presence of at least one of the referenced item.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe. Thus compositions slightly outside the cited ranges are also encompassed by the scope of the present claims.

An “active agent” is any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers in pure form and mixtures thereof are encompassed. In addition, compounds with carbon-carbon double bonds may occur in Z- and E-forms, with all isomeric forms of the compounds being included in the present invention. In these situations, the single enantiomers, i.e. optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

The terms “comprising,” “including,” and “containing” are non-limiting. Other non-recited elements may be present in embodiments claimed by these transitional phrases. Where “comprising,” “containing,” or “including” are used as transitional phrases other elements may be included and still form an embodiment within the scope of the claim. The open-ended transitional phrase “comprising” encompasses the intermediate transitional phrase “consisting essentially of” and the close-ended phrase “consisting of.”

“Alkyl” is a branched or straight chain saturated aliphatic hydrocarbon group, having the specified number of carbon atoms, generally from 1 to about 8 carbon atoms. The term C₁-C₆-alkyl as used herein indicates an alkyl group having from 1, 2, 3, 4, 5, or 6 carbon atoms. Other embodiments include alkyl groups having from 1 to 6 carbon atoms, 1 to 4 carbon atoms or 1 or 2 carbon atoms, e.g. C₁-C₈-alkyl, C₁-C₄-alkyl, and C₁-C₂-alkyl. Examples of alkyl include, but are not limited to, methyl, ethyl, n-propyl, isopropyl, n-butyl, 3-methylbutyl, t-butyl, n-pentyl, and sec-pentyl. In this disclosure “alkyl” includes alkyl groups as described in which one or more C—C saturated bonds is replace by a double or triple bonds, i.e. alkenyl or alkynyl groups.

“Aryl” indicates aromatic groups containing only carbon in the aromatic ring or rings. Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Aryl groups include, for example, phenyl, naphthyl, including 1-naphthyl, 2-naphthyl, and bi-phenyl. An “arylalkyl” substituent group is an aryl group as defined herein, attached to the group it substitutes via an alkylene linker. The alkylene is an alkyl group as described herein except that it is bivalent.

Carbocycle is a saturated, unsaturated or aromatic cyclic group having the indicated number of ring atoms, with all ring atoms being carbon. (Carbocycle)alkyl is a carbocycle, as defined, attached to the group it substitutes via an alkyl linker.

“Cycloalkyl” is a saturated hydrocarbon ring group, having the specified number of carbon atoms. Monocyclic cycloalkyl groups typically have from 3 to 6 (3, 4, 5, or 6) carbon ring atoms. Cycloalkyl substituents may be pendant from a substituted nitrogen, oxygen, or carbon atom, or a substituted carbon atom that may have two substituents may have a cycloalkyl group, which is attached as a spiro group. Examples of cycloalkyl groups include cyclopropyl, cyclobutyl, cyclopentyl, and cyclohexyl. (Cycloalkyl)alkyl is a cycloalkyl group as described attached to the group it substitutes via an alkyl group, such as a C₁-C₄alkyl or C₁-C₂alkyl.

Cₘₐₓ is the measured concentration of an active concentration in the plasma at the point of maximum concentration.

A “heteroalkyl” group is an alkyl group as described with at least one carbon replaced by a heteroatom, e.g. N, O, or S.

A “heteroaryl” group is a stable monocyclic aromatic ring having the indicated number of ring atoms which contains from 1 to 4, or in some embodiments from 1 to 2, heteroatoms chosen from N, O, and S, with remaining ring atoms being carbon, or a stable bicyclic or tricyclic system containing at least one 5- to 7-membered aromatic ring which contains from 1 to 4, or in some embodiments from 1 to 2, heteroatoms chosen from N, O, and S, with remaining ring atoms being carbon. Monocyclic heteroaryl groups typically have from 5 to 7 ring atoms. In certain embodiments there heteroaryl group is a 5- or 6-membered moncyclic heteroaryl group having 1, 2, 3, or 4 heteroatoms chosen from N, O, and S, with no more than 2 O atoms and 1 S atom. Examples of heteroaryl groups include thienyl, furanyl, oxazolyl, thiazolyl, imidazolyl, pyrrolyl, pyrazolyl, pyridyl, pyrimidinyl, and pyridizinyl groups.

A “patient” is a human or non-human animal in need of medical treatment. Medical treatment includes treatment of an existing condition, such as a disorder or injury. In certain embodiments treatment also includes prophylactic or preventative treatment, or diagnostic treatment.

“Pharmaceutical compositions” are compositions comprising at least one active agent, such as a compound or salt, solvate, or hydrate of Formula (I), and at least one other substance, such as a carrier. Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as a seizure disorder.

“Povidone” also known as polyvidone and polyvinylpyrrolidone (PVP) is a water soluble polymer made from the monomer, N-vinylpyrrolidone. Plasdone C-12 and C-17 are pharmaceutical grade homopolymers of N-vinylpyrrolidone. Plasdone C-12 has a K value of 10-2-13.8 and nominal molecular weight of 4000 d. Plasdone C-17 has a K-value of 15.5-17.5 and nominal molecular weight of 10,000 d.

The term “substituted” as used herein, means that any one or more hydrogens on the designated atom or group is replaced with a selection from the indicated group, provided that the designated atom's normal valence is not exceeded. When the substituent is oxo (i.e., ═O) then 2 hydrogens on the atom are replaced. When an oxo group substitutes a heteroaromatic moiety, the resulting molecule can sometimes adopt tautomeric forms. For example a pyridyl group substituted by oxo at the 2- or 4-position can sometimes be written as a pyridine or hydroxypyridine. Combinations of substituents and/or variables are permissible only if such combinations result in stable compounds or useful synthetic intermediates. A stable compound or stable structure is meant to imply a compound that is sufficiently robust to survive isolation from a reaction mixture and subsequent formulation into an effective therapeutic agent. Unless otherwise specified, substituents are named into the core structure. For example, it is to be understood that aminoalkyl means the point of attachment of this substituent to the core structure is in the alkyl portion and alkylamino means the point of attachment is a bond to the nitrogen of the amino group.

Suitable groups that may be present on a “substituted” or “optionally substituted” position include, but are not limited to, e.g., halogen; cyano; —OH; oxo; —NH₂; nitro; azido; alkanoyl (such as a C₂-C₆ alkanoyl group); C(O)NH₂; alkyl groups (including cycloalkyl and (cycloalkyl)alkyl groups) having 1 to about 8 carbon atoms, or 1 to about 6 carbon atoms; alkenyl and alkynyl groups including groups having one or more unsaturated linkages and from 2 to about 8, or 2 to about 6 carbon atoms; alkoxy groups having one or more oxygen linkages and from 1 to about 8, or from 1 to about 6 carbon atoms; aryloxy such as phenoxy; alkylthio groups including those having one or more thioether linkages and from 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups including groups having one or more N atoms and from 1 to about 8, or from 1 to about 6 carbon atoms; mono- or dialkylamino groups including groups having alkyl groups from 1 to about 6 carbon atoms; mono- or dialkylaminocarbonyl groups (i.e. alkylNHCO— or (alkyl₁)(alkyl₂)NCO—) having alkyl groups from about 1 to about 6 carbon atoms; aryl having 6 or more carbons. In certain embodiments substituents that may be present at an optionally substituted position include halogen, hydroxyl, —CN, —SH, nitro, oxo, amino, C₁-C₆alkyl, C₁-C₆alkoxy, C₁-C₆alkylthio, C₁-C₆alkylsulfonyl, mono- and di-(C₁-C₄alkyl)amino, mono- and di-C₁-C₄alkylcarboxamide, (C₃-C₆cycloalkyl)C₀-C₂alkyl, C₁-C₂haloalkyl; and C₁-C₂haloalkoxy.

A “therapeutically effective amount” or “effective amount” is that amount of a pharmaceutical agent to achieve a pharmacological effect. The term “therapeutically effective amount” includes, for example, a prophylactically effective amount. An “effective amount” of neurosteroid is an amount needed to achieve a desired pharmacologic effect or therapeutic improvement without undue adverse side effects. The effective amount of neurosteroid will be selected by those skilled in the art depending on the particular patient and the disease. It is understood that “an effective amount” or “a therapeutically effective amount” can vary from subject to subject, due to variation in metabolism of neurosteroid, age, weight, general condition of the subject, the condition being treated, the severity of the condition being treated, and the judgment of the prescribing physician.

“Treat” or “treatment” refers to any treatment of a disorder or disease, such as inhibiting the disorder or disease, e.g., arresting the development of the disorder or disease, relieving the disorder or disease, causing regression of the disorder or disease, relieving a condition caused by the disease or disorder, or reducing the symptoms of the disease or disorder.

### Chemical Description

The disclosure includes injectable nanoparticle neurosteroid formulations. The neurosteroid may be a compound of Formula I. Formula I includes allopregnanolone, ganaxolone, alphaxalone, alphadolone, hydroxydione, minaxolone, pregnanolone, acebrochol, isopregnanolone, or tetrahydrocorticosterone.

Ganaxolone (CAS Reg. No. 38398-32-2, 3α-hydroxy, 3β-methyl-5α-pregnan-20-one) is a synthetic steroid with anti-convulsant activity useful in treating epilepsy and other central nervous system disorders.

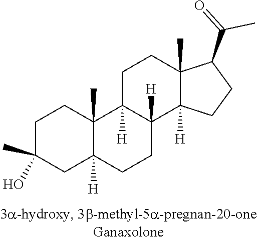

Ganaxolone has a relatively long half-life—approximately 20 hours in human plasma following oral administration (Nohria, V. and Giller, E., Neurotherapeutics, (2007) 4(1): 102-105). Furthermore, ganaxolone has a short Tₘax, which means that therapeutic blood levels are reached quickly. Thus initial bolus doses (loading doses) may not be required, which represents an advantage over other treatments. Ganaxolone is useful for treating seizures in adult and pediatric epileptic patients.

Allopregnanolone (CAS Reg. No. 516-54-1, 3α,5α-tetrahydroprogesterone) is an endogenous progesterone derivative with anti-convulsant activity.

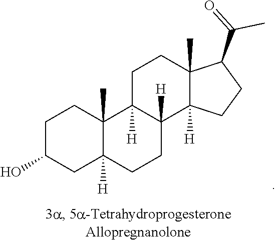

Allopregnanolone has a relatively short half-life, about 45 minutes in human plasma. In addition to its efficacy in treating seizures, allopregnanolone is being evaluated for use in treating neurodegenerative diseases including Alzheimer's disease, Parkinson's disease, Huntington's disease, and amyotrophic lateral sclerosis and for treating lysosomal storage disorders characterized by abnormalities in cholesterol synthesis, such as Niemann Pick A, B, and C, Gaucher disease, and Tay Sachs disease. (See U.S. Pat. No. 8,604,011, which is hereby incorporated by reference for its teachings regarding the use of allopregnanolone for treating neurological disorders.)

Alphaxalone, also known as alfaxalone, (CAS Reg. No. 23930-19-0, 3α-hydroxy-5α-pregnan-11, 20-dione) is a neurosteroid with an anesthetic activity. It is used as a general anaesthetic in veterinary practice. Anaesthetics are frequently administered in combination with anti-convulsants for the treatment of refractory seizures. An injectable nanoparticle neurosteroid dosage form containing alphaxalone alone or in combination with either ganaxolone or allopregnanolone is within the scope of this disclosure.

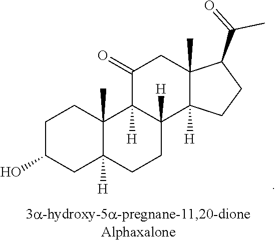

Alphadolone, also known as alfadolone, (CAS Reg. No. 14107-37-0, 3α, 21-dihydroxy-5α-pregnan-11, 20-dione) is a neurosteroid with anaesthetic properties. Its salt, alfadolone acetate is used as a veterinary anaesthetic in combination with alphaxalone.

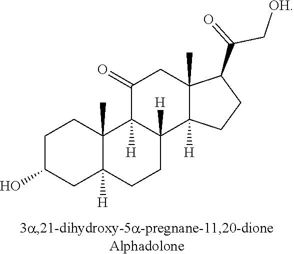

Additional neurosteroids that may be used in the injectable nanoparticle neurosteroid formulation of this disclosure include formulations include hydroxydione (CAS Reg. No. 303-01-5. (5β)-21-hydroxypregnane-3,20-dione), minaxolone (CAS Reg. No. 62571-87-3, 2β,3α,5α,11α)-11-(dimethylamino)-2-ethoxy-3-hydroxypregnan-20-one), pregnanolone (CAS Reg. No. 128-20-1, (3α,5β)-d-hydroxypreganan-20-one), renanolone (CAS Reg. No. 565-99-1, 3α-hydroxy-5β-pregnan-11,20-dione), isopregnanolone (CAS Reg. No. 516-55.2, 3β-Hydroxy-5α-pregnan-20-one), or tetrahydrocorticosterone (CAS Reg. No. 68-42-8, 3α,5α-pregnan-20-dione).

The disclosure includes compounds of Formula I as disclosed in the SUMMARY section in which the neurosteroid is compound of Formula I, where

R¹ is a group of the formula

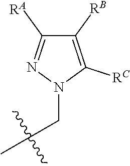

each instance of R^{A}, R^{B}, and R^{C} is, independently, hydrogen, halogen, —NO₂, —CN, —OR^{GA}, —N(R^{GA})₂, —C(═O)R^{GA}, —C(═O)OR^{GA}, —OC(═O)R^{GA}, —OC(═O)OR^{GA}, —C(═O)N(R^{GA})₂, —N(R^{GA})C(═O)R^{GA}, —OC(═O)N(R^{GA})₂, —N(R^{GA})C(═O)OR^{GA}, —N(R^{GA})C(═O)N(R^{GA})₂, —SR^{GA}, —S(O)R^{GA}, e.g., —S(═O)R^{GA}, —S(═O)₂R^{GA}, —S(═O)₂OR^{GA}, —OS(═O)₂R^{GA}, —S(═O)₂N(R^{GA})₂, —N(R^{GA})S(═O)₂R^{GA}, optionally substituted alkyl, optionally substituted C₃-C₆ carbocyclyl, or optionally substituted 3- to 6-membered heterocylyl; and where instance of R^{GA} is independently hydrogen, optionally substituted alkyl, optionally substituted C₃-C₆ carbocylyl, optionally substituted 3- to 6-membered heterocylyl, optionally substituted aryl, optionally substituted heteroaryl, an oxygen protecting group when attached to oxygen, nitrogen protecting group when attached to nitrogen, or two R^{GA} groups are taken with the intervening atoms to form a substituted or unsubstituted heterocylyl or heteroaryl ring.

In certain embodiments the neurosteroid is ganaxolone. In other embodiments the neurosteroid is allopregnanolone, alphaxalone, minaxolone, allotetrahydrodeoxycorticosterone, etiocholanone, dehydroepiandrosterone (including dehydroepiandrosterone sulfate), isopregnanolone, or pregnanolone (including pregnanolone sulfate).

In certain embodiments the neurosteroid is a compound of Formula I, as shown in the SUMMARY section, or a pharmaceutically acceptable salt of such a compound. In certain embodiments the neurosteroid is ganaxolone. In other embodiments the neurosteroid is allopregnanolone, alphaxalone, minaxolone, tetrahydrodeoxycorticosterone, etiocholanone, dehydroepiandrosterone, isopregnanolone, or pregnanolone, or a pharmaceutically acceptable salt of any of the foregoing.

The disclosure includes compounds of Formula I as disclosed in the SUMMARY section in which the neurosteroid is compound of Formula I, where any of the following conditions for the variables (e.g. R¹-R¹¹) are met. All definitions of the variables used in this disclosure can be combined so long as a stable compound of Formula I results.

R¹ is methyl, —CH₂Br, or —CH₂OH.

R¹ is a group of the formula

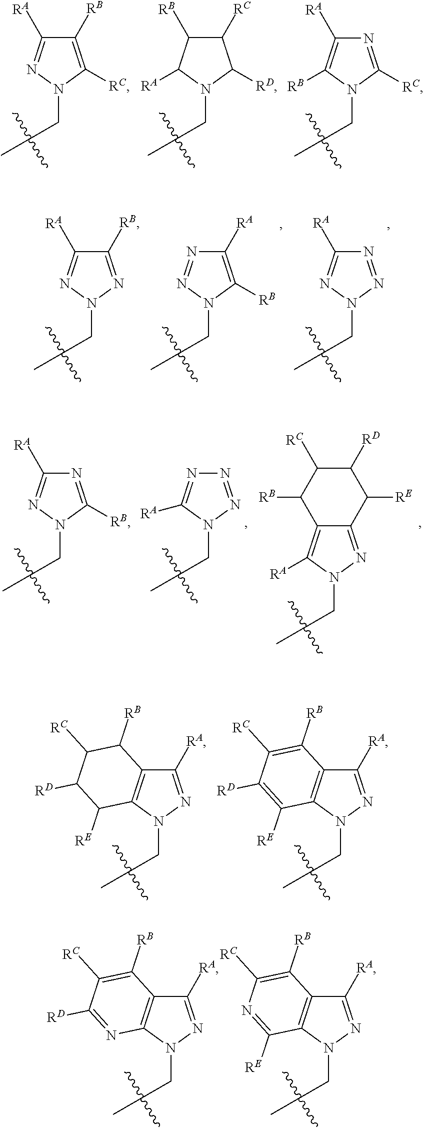

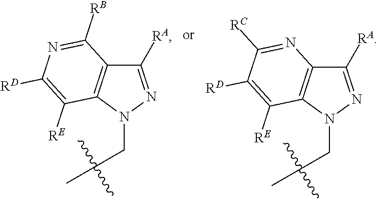

each instance of R^{A}, R^{B}, R^{C}, R^{D}, and R^{E} is, independently, hydrogen, halogen, —NO₂, —CN, —OR^{GA}, —N(R^{GA})₂, —C(═O)R^{GA}, —C(═O)OR^{GA}, —OC(═O)R^{GA}, —OC(═O)OR^{GA}, —C(═O)N(R^{GA})₂, —N(R^{GA})C(═O)R^{GA}, —OC(═O)N(R^{GA})₂, —N(R^{GA})C(═O)OR^{GA}, —N(R^{GA})C(═O)N(R^{GA})₂, —SR^{GA}, —S(O)R^{GA}, e.g., —S(═O)R^{GA}, —S(═O)₂R^{GA}, —S(═O)₂OR^{GA}, —OS(═O)₂R^{GA}, —S(═O)₂N(R^{GA})₂, —N(R^{GA})S(═O)₂R^{GA}, optionally substituted alkyl, optionally substituted C₃-C₆ carbocylyl, or optionally substituted 3- to 6-membered heterocylyl; and where instance of R^{GA} is independently hydrogen, optionally substituted alkyl, optionally substituted C₃-C₆ carbocylyl, optionally substituted 3- to 6-membered heterocylyl, optionally substituted aryl, optionally substituted heteroaryl, an oxygen protecting group when attached to oxygen, nitrogen protecting group when attached to nitrogen, or two R^{GA} groups are taken with the intervening atoms to form a substituted or unsubstituted heterocylyl or heteroaryl ring.

In certain embodiments R^{A}, R^{B}, R^{C}, R^{D} and R^{E} are independently chosen from hydrogen, halogen, cyano, methyl, methyoxy, ethyl, ethoxy, C₁-C₂haloalkyl, and C₁-C₂haloalkoxy.

In certain embodiments, R¹ is a group of the formula

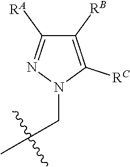

and R^{A}, R^{B}, and R^{C}, are all hydrogen or R^{A} and R^{C} are hydrogen and R^{B} is cyano.

The disclosure pertains to compounds and salts of Formula I having any of the above R¹ values where R² is methyl, R³ is hydrogen, R⁴ and R^{4a} are both hydrogen or are taken together to form an oxo group; R⁶ is hydrogen, R⁷ is hydrogen, R⁸ is hydrogen or methyl, R⁹ is hydroxyl, or R⁸ and R⁹ are taken together to form an oxo group, and R¹⁰ and R^{10A} are both hydrogen.

In certain embodiments [symbol] is also a single bond.

In certain embodiments a Formula I is represented by one of the following substructures:

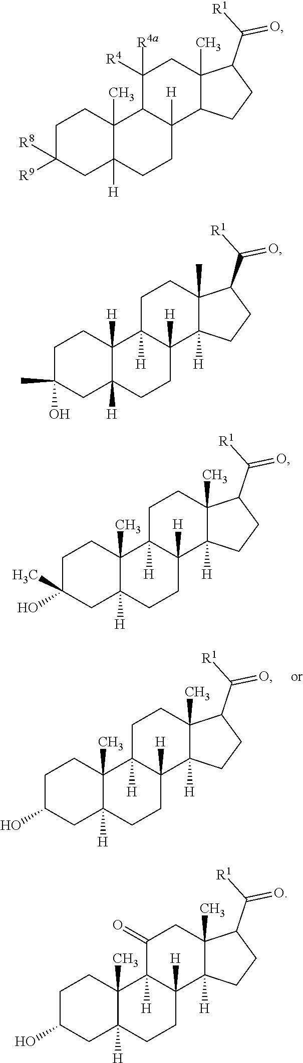

### Neurosteroid Particle Suspensions

This disclosure is directed to sustained release injectable neurosteroid formulations, including formulations suitable for intramuscular and subcutaneous administration. It has been surprisingly discovered that injectable suspensions of neurosteroid particles containing neurosteroid and at least one polymeric surface stabilizer provide high brain levels of neurosteroid. Suitable polymeric surface stabilizers include hydroxyethyl starch, dextran, and povidone. The injectable neurosteroid particles contain a neurosteroid of Formula I, a polymeric surface stabilizer, and a optionally a surfactant as a second surface stabilizer and provide an effective concentration of neurosteroid to the patient for at least 48 hours after injection, but in certain embodiments provide an effective concentration of neurosteroid to the patient for up to 4 weeks after injection. In certain embodiments the neurosteroid may be ganaxolone or allopregnanolone.

This disclosure is also directed to a sustained release injectable neurosteroid formulation comprising suspended particles containing a neurosteroid of Formula I, a first surface stabilizer, which is a polymer, and a second surface stabilizer which is a surfactant.

The disclosure provides sustained release injectable neurosteroid formulations, including formulations containing neurosteroid particles comprising a neurosteroid of Formula I, at least one polymeric surface stabilizer, such as hydroxyethyl starch, dextran, or povidone and a surfactant. In certain embodiments the nanoparticles comprise ganaxolone or allopregnanolone, hydroxyethyl starch, and a surfactant. Injectable neurosteroid nanoparticle formulations disclosed herein include formulations suitable for intravenous bolus, intravenous infusion, transdermal, intramuscular, subcutaneous injection, and intraperitoneal injection.

Many neurosteroids are very poorly soluble in water and thus difficult to formulate as aqueous injectable dosage forms. For example, ganaxolone is very poorly soluble in water (<0.001 mg/mL). The inventors have found that neurosteroids may be formulated as an aqueous injectable suspension by preparing the neurosteroid as a neurosteroid particle, the neurosteroid particle containing a polymeric surface stabilizer, such as either hydroxyethyl starch, dextran, or povidone, and an additional surface stabilizer, where the additional surface stabilizer is a surfactant.

The injectable neurosteroid formulation includes a polymeric surface stabilizer. In certain embodiment the surface stabilizer is a blood replacer, such as a blood volume expander. In certain embodiments the surface stabilizer is either hydroxyethyl starch, dextran, or povidone. Hydroxyethyl starch is used as a blood volume expander in patients suffering from severe blood loss. Grades of hydroxyethyl starch suitable for use in the neurosteroid nanoparticles include 130/0.4 (CAS Reg. No. 9005-27-0). In certain embodiments the surface stabilizer is dextran. Dextran is a single chain branched glucan having chains of varying lengths. Like hydroxyethyl starch, dextran is also used as a blood volume expander. Dextrans are classified according to MW. Dextrans having molecular weights from 40 kD to 75 kD have been used as blood volume expanders. Suitable dextrans for intravenous use include Dextran 40, Dextran 60, Dextran 70, and Dextran 75. In certain embodiments the surface stabilizer is a dextran having a molecular weight from about 40 kD to about 75 kD. In certain embodiments the surface stabilizer is Dextran 70. Povidone, also known as polyvinylpyrrolidone, is another approved plasma expander. Povidone includes PLASDONE C-12 and C-17 from Ashland, Inc.

Other excipients useful as surface stabilizers for the sustained release injectable neurosteroid formulation include human serum albumin, hydrolyzed gelatin, polyoxyethylene castor oil, polyoxyethylene hydrogenated castor oil, and polyethylene glycol (e.g., PEG 300 and PEG 400).

The sustained release injectable neurosteroid formulation can also include a surfactant. Surfactants include compounds such as lecithin (phosphatides), sorbitan trioleate and other sorbitan esters, polyoxyethylene sorbitan fatty acid esters (e.g., the commercially available TWEENS such as polyoxyethylene sorbitan monolaurate (TWEEN 20) and polyoxyethylene sorbitan monooleate (TWEEN 80) (ICI Speciality Chemicals)); poloxamers (e.g., poloxamer 188 PLURONIC F68 and poloxamer 338 (PLURONIC F108), which are block copolymers of ethylene oxide and propylene oxide), lecithin, sodium cholesterol sulfate or other cholesterol salts, and bile salts, such as sodium deoxycholate. Additional bile salts that may be used as surfactants include sodium cholate, sodium glycholate, salts of deoxycholic acid, salts of glycholic acid, salts of chenodeoxycholic acid, and salts of lithocholic acid.

The disclosure includes neurosteroid particles having a volume weighted median diameter (D50) of from 50 nm to 10 microns, 100 nm to 10 microns, 200 nm to 10 microns, 500 nm to 10 microns, 1 micron to 10 microns, 1 micron to 5 microns, 500 nm to 2000 nm, 50 nm 500 nm, 10 nm to 350 nm, or having a D50 of from 50 nm to 300 nm, or having a D50 of from 100 nm to 250 nm, or having a D50 of 150 nm to 220 nm, or having a D50 of less than 2000 nm, less than 500 nm, of less than 350 nm, less than 300 nm, less than 250 nm, or less than 200 nm. In one aspect the neurosteroid nanoparticles have at least one of the following properties: (a) greater than 90% of the neurosteroid by weight is in the form of a particle having an effective size of 500 nm to 5 microns; (b) at least 20% of the neurosteroid by weight is in the form of an amorphous powder; (c) at least 50% of the neurosteroid by weight is in the form of a crystalline powder of a single polymorph; (d) at least 50% of the neurosteroid is in the form of a semi-crystalline powder; (e) the neurosteroid is in the form of particles wherein at least 50%, or at least 60%, or at least 70%, or at least 80%, or at least 90% of the particles by weight have an effective size less than 10 microns; (f) the neurosteroid is in the form of particles wherein at least 50% of the particles by weight have an effective size less than 5 microns; (g) the neurosteroid is in the form of particles having a D50 of about 100 nm to about 5 microns, wherein the particle size distribution is described by a three-slice model in which a certain percentage has an effective particle size by weight between about 100 nm and about 500 nm, a certain percentage has an effective particle size by weight between about 500 nm and about 2000 nm, and a certain percentage has an effective particle size by weight above 2000 nm, and further wherein the three-slice model is identified as x %/y %/z %, respectively (e.g., 40%/30%/30%); (p) the neurosteroid has a three-slice distribution selected from the group 40%/30%/30%, 50%/30%/20%, 60%/30%/10%, 40%/40%/20%, 50%/40%/10%, 70%/20%/10%, 50%/45%/5%, 70%/25%/5%, 60%/35%/5%, 80%/15%/5%, 70%/30%/0%, 60%/40%/0%, 90%/10%/0%, and 100%/0%/0%; (h) the neurosteroid is in the for of particles, wherein standard deviation of the particle size distribution divided by the volume-weighted mean diameter is less than about 30%, less than about 25%, less than about 20%, less than about 15%, or less than about 10%. In alternative embodiments, the neurosteroid in the composition has at least two of the aforementioned properties; at least about three of the aforementioned properties; at least about four of the aforementioned properties; or at least five of the aforementioned properties.

The neurosteroid nanoparticles may be prepared by grinding. Grinding can take place in any suitable grinding mill. Suitable mills include an air jet mill, a roller mill, a ball mill, an attritor mill, a vibratory mill, a planetary mill, a sand mill and a bead mill. A high energy media mill is preferred when small particles are desired. The mill can contain a rotating shaft.

The preferred proportions of the grinding media, neurosteroid, the optional liquid dispersion medium, and dispersing, wetting or other particle stabilizing agents present in the grinding vessel can vary within wide limits and depends, for example, the size and density of the grinding media, the type of mill selected, the time of milling, etc. The process can be carried out in a continuous, batch or semi-batch mode. In high energy media mills, it can be desirable to fill 80-95% of the volume of the grinding chamber with grinding media. On the other hand, in roller mil is, it frequently is desirable to leave the grinding vessel up to half filled with air, the remaining volume comprising the grinding media and the liquid dispersion media, if present. This permits a cascading effect within the vessel on the rollers which permits efficient grinding. However, when foaming is a problem during wet grinding, the vessel can be completely filled with the liquid dispersion medium or an anti-foaming agent may be added to the liquid dispersion.

The attrition time can vary widely and depends primarily upon the drug, mechanical means and residence conditions selected, the initial and desired final particle size and so forth.

After attrition is completed, the grinding media is separated from the milled neurosteroid particulate product (in either a dry or liquid dispersion form) using conventional separation techniques, such as by filtration, sieving through a mesh screen, and the like.

In one aspect, the grinding media comprises beads having a size ranging from 0.05-4 mm, preferably 0.1-0.4 mm. For example, high energy milling of neurosteroid with yttrium stabilized zirconium oxide 0.4 mm beads for a milling residence time of 25 minutes to 1.5 hours in recirculation mode at 2500 RPM. In another example, high energy milling of neurosteroid with plastic beads (e.g. Purolite® Puromill 300) for a mil ling time of 400 minutes in recirculation mode at 4200 RPM. In another example, high energy milling of neurosteroid with 0.1 mm zirconium oxide balls for a milling residence time of 2 hours in batch mode. Additionally, the milling temperature should not exceed 50° C. as the viscosity of the suspension may change dramatically. The milling concentration is from about 1% to about 40% neurosteroid by weight. In one embodiment, the concentration is 25% neurosteroid by weight. In one embodiment, the milling media contains at least one agent to adjust viscosity so that the desired particles are suspended evenly, and a wetting and/or dispersing agent to coat the initial neurosteroid suspension so a uniform feed rate may be applied in continuous milling mode. In another embodiment, batch milling mode is utilized with a milling media containing at least one agent to adjust viscosity and/or provide a wetting effect so that the neurosteroid is well dispersed amongst the grinding media.

### Sustained Release Injectable Neurosteroid Formulations

The disclosure provides sustained release injectable neurosteroid formulations containing the neurosteroid at a concentration of about 0.25 mg/mL, about 0.5 mg/mL, about 1.0 mg/mL, about 1.5 mg/mL, about 2.0 mg/mL, about 2.5 mg/mL, about 3.0 mg/mL, about 3.5 mg/mL, about 4.0 mg/mL, about 4.5 mg/mL, about 5.0 mg/mL, about 5.5 mg/mL, about 6.0 mg/mL, about 6.5 mg/mL, about 7.0 mg/mL, about 7.5 mg/mL, about 8.0 mg/mL, about 8.5 mg/mL, about 9.0 mg/mL, about 10 mg/mL, about 11 mg/mL, about 12 mg/mL, about 13 mg/mL, or about 15 mg/mL, or about 25 mg/ml, about 50 mg/ml, about 75 mg/ml, about 100 mg/ml, about 150 mg/ml, about 200 mg/ml and about 250 mg/ml. All ranges including any two of the foregoing concentrations of neurosteroid as endpoints are also included in the disclosure. For example, the disclosure includes neurosteroid nanoparticle formulations containing from about 0.5 mg/mL to about 15 mg/mL, about 1.0 mg/mL to about 10 mg/mL, about 2.0 mg/mL to about 8.0 mg/mL, or about 4.0 mg/mL to about 8.0 mg/mL neurosteroid, or about 25 mg/ml to about 250 mg/ml, or about 100 mg/ml to 250 mg/ml, or about 25 mg/ml to 150 mg/ml.

The neurosteroid particles include neurosteroid and a polymeric surface stabilizer, such as either hydroxyethyl starch, povidone, or dextran, in a weight to weight ratio of neurosteroid to polymeric surface stabilizer is about 10:1 to 0.2:1, or about 5:1 to about 0.5:1, or about 4:1 to about 1:1, or about 3.5:1 to about 3:1, or about 3.3:1.

The disclosure includes embodiments in which the sustained release injectable neurosteroid formulation additionally comprises a buffer. In certain embodiments the buffer is a phosphate buffer. In certain embodiments the buffer is phosphate buffered saline.

The sustained release injectable neurosteroid formulations may also include an acid or base buffer to adjust pH to desired levels. In some embodiments the desired pH is 2.5-11.0, 3.5-9.0, or 5.0-8.0, or 6.0-8.0, or 7.0-7.6, or about 7.4. Examples of acid buffers useful in the injectable neurosteroid nanoparticle formulation include oxalic acid, maleic acid, fumaric acid, lactic acid, malic acid, tartaric acid, citric acid, benzoic acid, acetic acid, methanesulfonic acid, histidine, succinic acid, toluenesulfonic acid, benzenesulfonic acid, ethanesulfonic acid and the like. Acid salts of the above acids may be employed as well. Examples of base buffers useful in the formulation include carbonic acid and bicarbonate systems such as sodium carbonate and sodium bicarbonate, and phosphate buffer systems, such as sodium monohydrogen phosphate and sodium dihydrogen phosphate. The concentration of each component of a phosphate buffer system will be from about 10 mM to about 200 mM, or from about 20 mM to about 150 mM, or from about 50 mM to about 100 mM.

The disclosure includes embodiments in which the pH of the neurosteroid formulation is about 7.4.

The formulation may contain electrolytes, such as sodium or potassium. The disclosure includes embodiments in which the formulation is from about 0.5% to about 1.5% sodium chloride (saline).

The formulation may contain tonicity adjusting agents so that it is isotonic with human plasma. Examples of tonicity adjusting agents useful in the formulation include, but are not limited to, dextrose, mannitol, sodium chloride, or glycerin. In certain embodiments the tonicity agent is 0.9% sodium chloride.

The sustained release injectable neurosteroid formulations may contain any pharmaceutically acceptable excipient compatible with the neurosteroid and capable of providing the desired pharmacological release profile for the dosage form. “Release profile” is the graph of neurosteroid concentration in plasma, blood, or a specified tissue, such as brain, versus time after administration. For example, certain sustained release dosage forms of this disclosure are “depot” formulations having a release profile in which the neurosteroid reaches atherapeutically effective concentration in plasma soon after injection (within 1-2 hours) and remains at an effective plasma concentration for at least 48 hours, or for a longer period such as at least 72 hours, 96 hours, 120 hours, 1 week, 2 weeks, 3 weeks, 4 weeks, or 1 month. The plasma levels also remain at all times below a threshold level as which side effects become unacceptable. The features of plasma levels above a minimal therapeutically effective concentration for an extended period but below the level at which side effects are unacceptable are the criteria of a depot, or sustained release formulation. Excipients include, for example, suspending agents, surfactants, solubilizers, stabilizers, lubricants, wetting agents, anti-foaming agent, diluents, and the like. Pharmaceutically acceptable excipients may comprise, but are not limited to, acacia, gelatin, colloidal silicon dioxide, calcium glycerophosphate, calcium lactate, maltodextrin, glycerin, magnesium silicate, polyvinylpyrrolidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

Suitable antifoaming agents include dimethicone, myristic acid, palmitic acid, and simethicone.

The sustained release injectable neurosteroid formulation may also contain a non-aqueous diluent such as ethanol, one or more polyol (e.g. glycerol, propylene glycol), an oil carrier, or any combination of the foregoing.

The sustained release injectable neurosteroid formulation may additionally comprise a preservative. The preservative may be used to inhibit bacterial growth or prevent deterioration of the active agent. Preservatives suitable for parenteral formulations include ascorbic acid, acetylcysteine, benzalkonium chloride, benzethonium chloride, benzoic acid, benzyl alcohol, chlorbutanol, chlorhexidene, m-cresol, 2-ethoxyethanol, human serum albumin, monothioglycerol, parabens (methyl, ethyl, propyl, butyl, and combinations), phenol, phenylmercurate salts (acetate, borate nitrate), sorbic acid, sulfurous acid salts (bisulfite and metabisulfite), and thimerosal. In certain embodiments the preservative is an antioxidants such ascorbic acid, glutathione, or an amino acid. Amino acids useful as antioxidants include methionine, cysteine, and L-arginine.

### Lyophilized Sustained Release Neurosteroid Formulations

The disclosure includes lyophilized forms of all formulations disclosed herein.

The sustained release injectable neurosteroid formulations provided in this disclosure are aqueous formulations or powder formulations including lyophilized forms, which may be readily resuspended in an aqueous solution to provide an injectable formulation. The disclosure includes embodiments in which the lyophilized neurosteroid powder comprises the neurosteroid, a surface stabilizer such as either hydroxyethyl starch or dextran, and a surfactant, wherein the injectable formulation is about 0.5% to about 90% neurosteroid, about 0.5% to about 80% neurosteroid, 0.5% to about 70% neurosteroid, 0.5% to about 60% neurosteroid, 0.5% to about 50% neurosteroid, 0.5% to about 40% neurosteroid, 0.5% to about 30% neurosteroid, 0.5% to about 20% neurosteroid, about 0.5% to about 10% neurosteroid, about 0.5% to about 2.0%, or about 1.0% to about 1.5% weight neurosteroid.

In certain embodiments the sustained release neurosteroid formulation is a lyophilized form that is dissolved in water or an aqueous solution prior to administration.

The lyophilized form may additionally include an antifoaming agent, a buffer (or pH adjuster), a cryoprotectant, a bulking agent, a tonicity adjuster, or a combination of any of the foregoing.

Bulking agents are useful for lyophilized formulation in which a low concentration of the active ingredient, or in the present case, in which a low concentration of the neurosteroid particle, is present. Bulking agents include mannitol, lactose, sucrose, trehalose, sorbitol, glucose, rafinose, glycine, histidine, polyethylene glycol (PEG), and polyvinyl pyrrolidone (PVP).

The removal of the hydration shell from an active agent during lyophilization can be destabilizing. In certain embodiments the lyophilized form contains a stabilizer which serves as a cryoprotectant. Stabilizers include agents which maintain a desirable attribute of the formulation over a time interval including but not limited to mechanical, chemical and temperature stressing that can be tested in a laboratory setting. Such attributes include stable particle size or homogeneity resulting in concentrations consistent with the labeled potency and maintaining purity.

Suitable cryoprotectant stabilizers include sugars such as sucrose, trehalose, glucose, rafinose, lactose, mannitol, sorbitol, histidine, polyethylene glycol (PEG), and polyvinyl pyrrolidone and sodium chloride.

### Ebeam Sterilized Formulations

Electron beam sterilization (ebeam) is a process using beta radiation, usually of high energy, to effect sterilization of a sample. Lyophilized powders of the sustained release injectable neurosteroid formulations may also be sterilized with ebeam radiation without adverse effects.

The sustained release injectable neurosteroid formulations of this disclosure can be subjected to ebeam irradiation, preferably at ambient temperature. This temperature remains relatively constant during irradiation.

The ebeam radiation is applied in an amount sufficient to destroy substantially all of the microbial contamination in the formulation. The total amount of ebeam radiation that the formulation is exposed to has been experimentally verified to: (1) show only a modest increase in particle size on storage following exposure to ebeam irradiation, (2) maintain the integrity of the particulate active agent, and (3) to show acceptable impurity concentrations following ebeam irradiation. The application of the ebeam radiation does not significantly degrade the neurosteroid or reduce its efficacy. The present disclosure enables products which meet cGMP requirements for sterile products without harming the neurosteroid nanoparticles.

In certain embodiments the ebeam radiation is applied in a cumulative amount of 25 kGray. Generally, the ebeam radiation will normally be applied in a range of 5 kGray to 50 kGray, 5 kGray to 40 kGray, 10 kGray to 30 kGray, 5 to 15 kGray, or 5 to 10 kGray. Multiple doses of radiation can be utilized to achieve a desired cumulative radiation dosage.

The microbial contamination which is to be destroyed is generally that of bacterial contamination and mycoplasma contamination.

Following sterilization the sustained release injectable neurosteroid formulations exhibit good overall stability, maintaining the pre-sterilized physical and chemical properties, while meeting cGMP requirements for sterility. The overall stability of the ebeam irradiated injectable formulations of neurosteroid is measured in terms of neurosteroid particle size, content of degradation products, and viscosity. Ebeam irradiation of the injectable neurosteroid formulations does not significantly alter the particle size of the neurosteroid particles. This is significant because if the sterilized product formed aggregates or large crystals, the formulation would no longer be useful as an injectable formulation.

### Methods of Treatment

The disclosure includes methods of treating seizure disorders including methods of treating epilepsy, genetic epilepsy disorders, including but not limited to PCDH19 female pediatric epilepsy, SCN8A epilepsy, catamenial epilepsy, seizures due Angelman syndrome, benign epilepsy with centro-temporal spikes (BECTS), seizures due to CDKL5 disorder, autosomal dominant nocturnal frontal lobe epilepsy (ADNFE), absence epilepsy, childhood absence epilepsy, Doose syndrome, Dravet syndrome, early myoclonic epilepsy (EME), epilepsy with generalized tonic-clonic seizures, epilepsy with myoclonic-absenses, infantile spasms (West's syndrome), seizures due to Landau-Kleffner syndrome, Lennox-Gastaut syndrome, epilepsy with myoclonic absences, frontal lobe epilepsy, juvenile myoclonic epilepsy, Lyfora progressive myoclonus epilepsy, Ohtahara syndrome, Panayiotopoulos syndrome, Rasmussen's syndrome, progressive myoclonic epilepsies, Ring chromosome 20 syndrome, temporal lobe epilepsy, epilepsy of infancy with migrating focal seizures, and other seizure disorders comprising administering an effective amount of the sustained release injectable neurosteroid formulation to a patient suffering from any of these seizure disorders.

The disclosure further includes methods of treating seizures arising from neurodegenerative disorders. Such neurodegenerative disorders include Parkinson's disease, Alzheimer's disease, Amyotrophic Lateral Sclerosis, and Huntington's disease. The disclosure includes methods of treating seizure arising from inflammatory disorders, such as multiple sclerosis. The disclosure includes methods of treating seizure disorders arising from lysosomal storage disorders including Neimann-Pick-C, Tay Sachs, Batten, Sandhoff, and Gaucher disease.

The disclosure also includes methods of using a sustained release injectable neurosteroid formulation of the disclosure to treat traumatic brain injury and stroke comprising administering an effective amount of the formulation to a patient suffering from recent traumatic brain injury or a recent stroke.

The disclosure also includes methods of using s sustained release injectable neurosteroid formulation of the disclosure to treat the symptoms of Angelman's syndrome and Fragile X syndrome.

The disclosure also provides a method of treating a CNS disorder or condition, including anxiety, generalized anxiety disorder, social anxiety disorder, panic disorder, anxiety or agitation due to Alzheimer's dementia, schizophrenia, substance withdrawal syndrome (alcohol, benzodiazepine, barbiturate, and cocaine), post traumatic stress disorder (PTSD), tremors, essential tremor, spasticity due to cerebral palsy, depression, (including major depression, major depressive disorder, severe depression, unipolar depression, unipolar disorder, or recurrent depression), postnatal or postpartum depression, atypical depression, melancholic depression, Psychotic Major Depression (PMD), catatonic depression, Seasonal Affective Disorder (SAD), dysthymia, double depression, Depressive Personality Disorder (DPD), Recurrent Brief Depression (RBD), minor depressive disorder, bipolar disorder or manic depressive disorder, post-traumatic stress disorders, post menopausal depression, depression caused by chronic medical conditions, treatment-resistant depression, refractory depression, suicidality, suicidal ideation, or suicidal behavior. In some embodiments, the depression is severe depression) premenstrual syndrome, severe premenstrual dysphoric disorder, and postpartum depression. This disclosure also includes a method of treating headaches including migraine headaches. This disclosure also includes a method of treating hypertension. The disclosure includes the following particular embodiments: a method of treating major depressive disorder, a method of treating postpartum depression.

The sustained release injection may be administered intramuscularly or subcutaneously. The dose of the single injection is sufficient to produce plasma level at steady state of at least 20 ng/ml, at least 50 ng/ml, of 10 ng/ml to 1000 ng/ml, 100 ng/mL to about 800 ng/mL, 10 ng/ml to 500 ng/ml, 10 ng/ml to 200 ng/ml, of 20 ng/ml to 100 ng/ml, or 40 ng/ml to 80 ng/ml, or at least 10 ng/ml for a period of 1 week, 2 weeks, 3 weeks, 4 weeks, or 1 month.

The amount of pregnane neurosteroid injected may be from about 1 mg/kg to about 750 mg/kg, from 2 mg/kg to 500 mg/kg, from 2 mg/kg to 250 mg/kg, mg/kg, from 5 mg/kg to 100 mg/kg, or from 5 mg/kg to 50 mg/kg. Methods of treatment also include administering multiple injections of the neurosteroid nanoparticle injectable formulation over a period of 1 to 10 days. The injections may be given at intervals of 1 to 24 hours. Dosing schedules in which the injectable neurosteroid nanoparticle formulation is injected every 48 hours, 72 hours, 1 week, 2 weeks, 3 weeks, 4 weeks, or 1 month are included herein.

In some embodiments, the pregnane neurosteroid is first administered as an intravenous dose, and the patient is subsequently administered the sustained release injectable neurosteroid. This allows the patient to be stabilized in a clinical setting, usually in an in-patient hospital setting, and the patient is then administered the sustained release injectable neurosteroid formulation allowing care to be continued in an outpatient setting. In certain embodiments the neurosteroid nanoparticle injectable formulation is administered intramuscularly or intravenously.

The disclosure includes embodiments in which the sustained release injectable neurosteroid formulation is administered to the patient as a single injection. In certain embodiments the single injection provides a sufficient amount of neurosteroid to provide a plasma level of neurosteroid of about 10 ng/ml to about 100 ng/mL to about 1000 ng/mL in the patient for at least 48 hours, or in some embodiments at least 72 hours, at least 96 hours, at least 120 hours, at least 1 week, at least 2 weeks, at least 3 weeks, at least 4 weeks, or at least 1 month.

The disclosure includes embodiments in which the neurosteroid formulation is administered as a single injection and the injection comprises from about 0.5 mg/kg to about 20 mg/kg neurosteroid. Or, optionally the single bolus dose comprises from about 2 mg/kg to about 15 mg/kg neurosteroid, or about 4 mg/kg to about 10 mg/kg neurosteroid, or from about 1 mg/kg to about 30 mg/kg neurosteroid.

### Combination Treatment

The disclosure includes embodiments in which the neurosteroid is the only active agent and embodiments in which the neurosteroid is administered in combination with one or more additional active agents. When used in combination with an additional active agent the neurosteroid and the additional active agent may be combined in the same formulation or may be administered separately. The neurosteroid may be administered while the additional active agent is being administered (concurrent administration) or may be administered before or after the additional active agent is administered (sequential administration).

The disclosure includes embodiments in which the additional active agent is an anti-convulsant. Anticonvulsants include GABA_{A} receptor modulators, sodium channel blocker, GAT-1 GABA transporter modulators, GABA transaminase modulators, voltage-gated calcium channel blockers, and peroxisome proliferator-activated alpha modulators.

The neurosteroid nanoparticle injectable formulation of this disclosure may be administered with another anticonvulsant agent. Anticonvulsants that may be used in combination with the neurosteroid nanoparticle injectable formulation of this disclosure include aldehydes, such as paraldehyde; aromatic allylic alcohols, such as stiripentol; barbiturates, including those listed above, as well as methylphenobarbital and barbexaclone; benzodiazepines include alprazolamn, bretazenil, bromazepam, brotizolam, chioridazepoxide, cinolazepam, clonazepam, chorazepate, clopazam, clotiazepam, cloxazolam, delorazepam, diazepam, estazolam, etizolam, ethyl loflazepate, flunitrazepam, flurazepam, flutoprazepam, halazepam, ketazolam, loprazolam, lorazepam, lormetazepam, medazepam, midazolam, nimetazepam, nitrazepam, nordazepam, oxazepam, phenenazepam, pinazepam, prazepam, premazepam, pyrazolam, quazepam, temazepam, tatrazepam, and triazolam; bromides, such as potassium bromide; carboxamides, such carbamazepine, oxcarbazepine, and eslicarbazepine acetate; fatty acids, such as valproic acid, sodium valproate and divalproex sodium; fructose derivatives, such as topiramate; GABA analogs such as gabapentin and pregabalin, hydantoins, such as ethotoin, phenytoin, mephenytoin, and fosphenytoin; other neurosteroids, such as allopregnanolone, oxasolidinediones, such as paramethadione, trimethadione, and ethadione, propionates such as beclamide; pyrimidinediones such as primidone, pyrrolidines such as brivaracetam, levetiracetam, and seletracetam, succinimides, such as ethosuximide, pensuximide, and mesuximide; sulfonamides such as acetazoloamide, sultiame, methazolamide, and zonisamide; triazines such as lamotrigine, ureas such as pheneturide and phenacenmide; NMDA antagonists, such as felbamate, and valproylamides such as valpromide and valnoctamide; and perampanel.

### SPECIFIC EMBODIMENTS

The disclosure provides the following specific embodiments that are further illustrated by the examples that follow.

### Specific Embodiment 1

A sustained release injectable neurosteroid formulation comprising neurosteroid particles having a D50 of less than 10 microns, the neurosteroid particles comprising

a) a neurosteroid of Formula I:

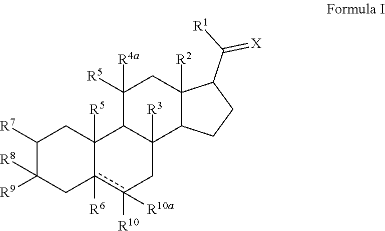

or a pharmaceutically acceptable salt thereof, wherein:

[symbol] is a double or single bond;

X is O, S, or NR¹¹;

R¹ is hydrogen, hydroxyl, optionally substituted alkyl, optionally substituted heteroalkyl, optionally substituted heteroaryl, optionally substituted heteroarylalkyl, optionally substituted aryl, or optionally substituted arylalkyl;

R⁴ is hydrogen, optionally substituted alkyl, optionally substituted cycloalkyl, or optionally substituted (cycloalkyl)alkyl, or —OR⁴⁰, where R⁴⁰ is hydrogen, optionally substituted alkyl, optionally substituted cycloalkyl, or optionally substituted (cycloalkyl)alkyl, or optionally substituted C₃-C₆carbocycle;

R^{4a} is hydrogen or R⁴ and R^{4a} are taken together to form an oxo (═O) group;

R², R³, R⁵, and R⁶, are each independently hydrogen, hydroxyl, halogen, optionally substituted alkyl, optionally substituted cycloalkyl, or optionally substituted (cycloalkyl)alkyl, or optionally substituted heteroalkyl;

R⁷ is hydrogen, halogen, optionally substituted alkyl, optionally substituted C₃-C₆carbocycle, optionally substituted (C₃-C₆carbocycle)alkyl or —OR⁷⁰ where R⁷⁰ is hydrogen, optionally substituted alkyl, optionally substituted C₃-C₆carbocycle, or optionally substituted (C₃-C₆carbocycle)alkyl;

R⁸ is hydrogen, optionally substituted alkyl or optionally substituted C₃-C₆carbocycle, and R⁹ is hydroxyl; or

R⁸ and R⁹ are taken together to form an oxo group;

R¹⁰ is hydrogen, halogen, hydroxyl, optionally substituted alkyl, optionally substituted heteroalkyl, optionally substituted C₃-C₆carbocyle, or optionally substituted (C₃-C₆carbocycle)alkyl, and R^{10a} is hydrogen, halogen, or optionally substituted alkyl, provided that if [symbol] is a double bond R^{10a} is absent;

each alkyl is a C₁-C₁₀alkyl and optionally contains one or more single bonds replaced by a double or triple bond;

each heteroalkyl group is an alkyl group in which one or more methyl group is replaced by an independently chosen —O—, —S—, —N(R¹¹)—, —S(═O)— or —S(═O)₂—, where R¹¹ is independently chosen at each occurrence and is hydrogen, alkyl, or alkyl in which one or more methylene group is replaced by —O—, —S—, —NH, or —N-alkyl; and

b) at least one polymeric surface stabilizer;

wherein the formulation provides an effective plasma concentration of the compound at steady state for at least 48 hours.

### Specific Embodiment 2

The sustained release injectable neurosteroid formulation of specific embodiment 1, wherein

R¹ is a group of the formula

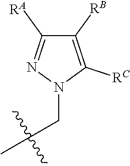

each instance of R^{A}, R^{B}, and R^{C} is, independently, hydrogen, halogen, —NO₂, —CN, —OR^{GA}, —N(R^{GA})₂, —C(═O)R^{GA}, —C(═O)OR^{GA}, —OC(═O)R^{GA}, —OC(═O)OR^{GA}, —C(═O)N(R^{GA})₂, —N(R^{GA})C(═O)R^{GA}, —OC(═O)N(R^{GA})₂, —N(R^{GA})C(═O)OR^{GA}, —N(R^{GA})C(═O)N(R^{GA})₂, —SR^{GA}, —S(O)R^{GA}, e.g., —S(═O)R^{GA}, —S(═O)₂R^{GA}, —S(═O)₂OR^{GA}, —OS(═O)₂R^{GA}, —S(═O)₂N(R^{GA})₂, —N(R^{GA})S(═O)₂R^{GA}, optionally substituted alkyl, optionally substituted C₃-C₆ carbocyclyl, or optionally substituted 3- to 6-membered heterocyclyl; and where instance of R^{GA} is independently hydrogen, optionally substituted alkyl, optionally substituted C₃-C₆ carbocyclyl, optionally substituted 3- to 6-membered heterocyclyl, optionally substituted aryl, optionally substituted heteroaryl, an oxygen protecting group when attached to oxygen, nitrogen protecting group when attached to nitrogen, or two R^{GA} groups are taken with the intervening atoms to form a substituted or unsubstituted heterocyclyl or heteroaryl ring.

### Specific Embodiment 3

The formulation of specific embodiment 1, wherein the neurosteroid is allopregnanolone, ganaxolone, alphaxalone, alphadolone, hydroxydione, minaxolone, pregnanolone, acebrochol, tetrahydrocorticosterone, isopregnanolone, or a compound of the formula

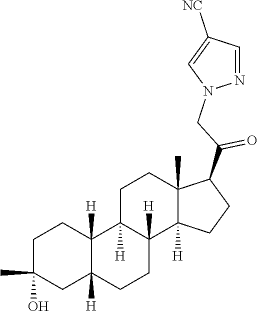

or a pharmaceutically acceptable salt of any of the foregoing.

### Specific Embodiment 4

The formulation of any one of specific embodiments 1 to 3, wherein the polymeric surface stabilizer is hydroxyethyl starch, dextran, povidone, or a mixture of any the foregoing.

### Specific Embodiment 5

The formulation of any one of specific embodiments 1 to 4, wherein the formulation comprises an additional surface stabilizer and the additional surface stabilizer is an ionic or nonionic surfactant; and an antifoaming agent.

### Specific Embodiment 6

The formulation of specific embodiment 4, wherein the polymeric surface stabilizer is hydroxyethyl starch; the surfactant is sodium cholate, sodium deoxycholate, sodium cholesterol sulfate, or a mixture of any of the foregoing; and the antifoaming agent is simethicone.

### Specific Embodiment 7

The formulation of any one of specific embodiments 1 to 5 additionally comprising a cryoprotectant, wherein the cryoprotectant is sucrose, dextrose, lactose, D-sorbitol, mannitol, or a mixture of any of the foregoing.

### Specific Embodiment 8

The formulation of any one of specific embodiments 1 to 6 additionally comprising one or more of the following

(a) 0.5% to 1.5% sodium chloride (weight percent);

(b) a buffer;

(c) a preservative, wherein the preservative is benzyl alcohol, chlorobutanol, 2-ethoxyethanol, parabens (including methyl, ethyl, propyl, butyl, and combinations), benzoic acid, sorbic acid, chlorhexidine, phenol, 3-cresol, thimerosal, a phenylmercurate salt, or a mixture of any of the foregoing.

### Specific Embodiment 9

The formulation of specific embodiment 1, wherein the neurosteroid is ganaxolone or allopregnanolone;

the at least one surface stabilizer is a polymeric surface stabilizer selected from hydroxyethyl starch, dextran, povidone, and a mixture of any of the foregoing, wherein the (wt:wt) ratio of the neurosteroid to the polymeric surface stabilizer is about 4:1 to about 0.2:1; and the formulation comprises an additional surface stabilizer and the additional surface stabilizer is a surfactant, selected from sodium deoxycholate, sodium cholesterol sulfate, and a mixture of any of the foregoing; wherein the ratio of neurosteroid to surfactant (w:w) is about 10:1.5 to about 10:0.1.

### Specific Embodiment 10

The formulation of any one of specific embodiments 1 to 9, wherein the formulation comprises neurosteroid particles have a D50 of 1 to 5 microns.

### Specific Embodiment 11

The formulation of any one of specific embodiments 1 to 10, wherein upon injection of the formulation into a patient the formulation release neurosteroid over a period of at least 48 hours, at least 72 hours, at least 96 hours, and least 120 hours, at least 1 week, at least 2 weeks, at least 3 weeks, or at least 4 weeks.

### Specific Embodiment 12

The formulation of any one of specific embodiments claims 1 to 11, wherein the formulation is an aqueous suspension comprising 1 mg/ml to 300 mg/ml neurosteroid, or 25 mg/ml to 150 mg/ml neurosteroid.

### Specific Embodiment 13

The formulation of specific embodiment 1, wherein the formulation is an aqueous formulation comprising

(a) neurosteroid particles having a D50 of 1 to 5 microns, the neurosteroid particles comprising ganaxolone, wherein the weight percent of the ganaxolone is 5 to 20%;

(b) a polymeric surface stabilizer selected from hydroxyethyl starch, dextran, and povidone, wherein the weight percent of the polymeric surface stabilizer is 2 to 50%;

(c) an additional surface stabilizer wherein the additional surface stabilizer is an ionic or nonionic surfactant selected from sodium cholate, sodium deoxycholate, and sodium cholesterol sulfate, wherein the weight percent of surfactant is 0.1% to 2.0%; and

(d) an antifoaming agent.

### Specific Embodiment 14

The formulation of specific embodiment 13, wherein the polymeric surface stabilizer is hydroxyethyl starch 130/0.4 and the weight percent of the polymeric surface stabilizer is about 5% to about 20%; and the additional surface stabilizer is sodium deoxycholate, wherein the weight percent of sodium deoxycholate is about 0.75%; and simethicone, wherein the weight percent of simethicone is 0.009%.

### Specific Embodiment 15

A method of treating a patient having seizures, comprising administering the formulation of any one of specific embodiments 1 to 14 to the patient.

### Specific Embodiment 16

A method of treating a patient having anxiety, agitation, depression, schizophrenia, post-traumatic stress disorder, or tremors, comprising administering the formulation of any one of specific embodiments 1 to 14 to the patient.

### Specific Embodiment 17

The method of specific embodiment 16, wherein the depression is post-partum depression.

### Specific Embodiment 18

The method of any one of specific embodiment 14 to 16, wherein the dosage of ganaxolone release from the formulation is from about 1 mg/kg/day to about 200 mg/kg/day.

### Specific Embodiment 19

The method of any one of specific embodiments 14 to 18, wherein the formulation provides a neurosteroid plasma concentration at steady state of at least 10 ng/ml for a period of at least 48 hours, 72 hours, 96 hours, 120 hours, 1 week, 2 weeks, 3 weeks or 4 weeks,

### Specific Embodiment 20

The method of specific embodiment 19, wherein the neurosteroid is ganaxolone and the formulation provides a ganaxolone plasma concentration at steady state of 100 ng/ml to 800 ng/ml for at least 1 week.

### EXAMPLES

### Abbreviations

### ALLO Allopregnanolone

### GNX Ganaxolone

HES Hydroxyethyl starch

### Example 1. Preparation of Ganaxolone Suspension (10% Wt) for Use in a Sustained Release Injectable Formulation Via Wet Bead Milling

An aqueous slurry (250 g) containing ganaxolone (25 g), hydroxyethyl starch (7.5 g), sodium deoxycholate (0.5 g) and 30% simethicone (1 drop) is milled using a Netzsch Mill (Minicer) with 0.3 mm YTZ beads (Yttrium stabilized grinding media, Tosoh Corporation, Japan, ZrO₂+HfO₂ (95 wt % (weight %)), Y₂O₂(5 wt %)). The particle size of the milled slurry is measured using a Horiba LA-910 laser diffraction particle size analyzer. Additional 250 g slurries are prepared containing the same amount of ganaxolone, sodium deoxycholate, and simethicone, but increasing concentrations of hydroxyethyl starch, e.g. from 10 g, 12.5 g, 15. g, 17.5 g, 20 g, 22.5 g, 25 g, 30 g, 35 g, 40 g, 45 g, or 50 g hydroxyethyl starch per 250 g slurry. The ratio of hydroxyethyl starch to ganaxolone ratio may be adjusted to obtain neurosteroid particles with desirable release profiles. For example the ratio of hydroxyethyl starch to ganaxolone may be 0.25:1 to 2:1. The batches are milled to obtain particles of varying sizes with different release profiles in plasma. The D50 particle size is measured on a Horiba 910 Laser Light Scattering instrument.

Table 1 shows the composition of additional ganaxolone/hydroxyethyl starch suspendable particles, for use in the sustained release injectable formulations.

TABLE 1

| Formulation | A | | B |
|---|---|---|---|
| Ganaxolone | 25 | g | 25 g |
| Hydroxyethyl starch | 25 g, 30 g, 35 g, 40 g, | | 25 g, 30 g, 35 g, 40 g, |
| | 45 g, 50 g | | 45 g, 50 g |
| Sodium deoxycholate | 3.0 | g | 3.0 |
| Simethicone (30%) | 0.15 | g | 0.05 |

### Example 2. Preparation of Ganaxolone Suspension Containing Dextran 70 Via Wet Bead Milling for Use in a Sustained Release Injectable Formulation

An aqueous ganaxolone slurry (250 g) containing ganaxolone (25 g), dextran 70 (7.5 g), sodium deoxycholate (1.5 g), and 30% simethicone (0.075 g) is milled using a Netzsch mill (Minicer) with 0.2 mm YTZ beads for 195 minutes to obtain a ganaxolone suspension with D50 of 159 nm (158 nm after 1 minute sonication). The same procedure is repeated for additional slurry batches using milling times of 40-200 minutes to obtain ganaxolone particles with different D50 values. Additional 250 g milling batches are prepared by this procedure that also contained ganaxolone (25 g), sodium deoxycholate (1.5 g), and 30% simethicone (0.075 g), and either 10 g, 12.5 g, 15 g, 17.5 g, 20 g, 22.5 g 25 g dextran 70.

### Example 3. Ganaxolone Suspension Containing Poloxamer 188

A KDL Bachofen Mill is configured with the batch chamber attachment (approx. 350 ml) and the 96 mm polyurethane rotor attached to the shaft. Next, 265 ml of 0.3 mm ytria-zirconia beads are added dry to the chamber, followed by 150 g of the Ganaxolone (GNX) slurry. Slowly, over 15 minutes, ganaxolone (50 g) is added to the milling media containing Pluronic F-68 (Poloxamer 188) with sustained stirring. The mixture is stirred slowly overnight. The slurry is milled at Speed 1 (1500 rpm) with intermittent measurement of particle size. The slurry is milled for 90 minutes. Additional 200 ml batches are produced having Pluronic F-68 concentrations of 12.5%, 15%, 17.5%, and 20% to produce particles of varying D50 particles sizes with different release profiles. The D50 measurement is measured on a Horiba 910 Laser Light Scattering instrument.

Methyl parabens or citric acid may also be added to the milling media. In addition, a combination of poloxamer 188 and dextran 70 may be used as the particle stabilizer.

Stabilizer Solution

| | | |
|---|---|---|
| Pluronic F-68 | 20.25 | g |
| Sodium deoxycholate | 2.03 | g |
| Simethicone emulsion 30% | 0.15 | g |
| Water (DI) | to 150 | g |

Ganaxolone Slurry

| | | |
|---|---|---|
| Ganaxolone | 50 | g |
| Stabilizer Solution | 150 | g |

Final Milling Composition (wt %)

| | |
|---|---|
| Ganaxolone | 25% |
| Pluronic F-68 | 10% |
| Deoxycholate | 1% |

### Example 4. Ganaxolone Suspension Containing Poloxamer 188, 0.1 Mm Beads

The KDL Bachofen mill is configured with the batch chamber attachment (approx. 350 ml) and the 96 mm polyurethane rotor attached to the shaft. Next, 300 ml of 0.1 mm ytria-zirconia beads are added dry to the chamber, followed by 134.6 gm Ganaxolone (GNX) slurry having the composition given in preceding Example 1. The slurry is milled for 60 minutes and the D50 particle size is measured after 20, 40, 60 minutes of milling.

| Time (min) | Particle size, μm | After sonication, μm |
|---|---|---|
| 20 | 0.182 | 0.183 |
| 40 | 0.164 | 0.165 |
| 60 | 0.162 | |

### Example 5. Bioavailability of Sustained Release Neurosteroid Formulations

The ganaxolone concentration in rat plasma and rat brain following administration of 9, 12, or 15 mg/kg ganaxolone as a sustained release hydroxyethyl starch 130/0.4 suspension is determined. Male Sprague-Dawley rats, 8-9 weeks of age, from Harlan Labs are used. Animals received food and water ad libitum throughout the study and are maintained on a 12 hr/12 hr light dark schedule with lights on at 7:00 AM. Animals are weighed prior to compound administration. Ganaxolone solutions are formulated at 2.5 mg/mL and the volume is adjusted to accommodate larger dosages. Injections are administered via the tail vein as a bolus dose.

Plasma is collected and 5, 15, 30 minutes post dosing and 1, 2, 4, 6, 12, 24, and 48 hours post dosing. Brains are collected at 5, 30, and 120 minutes post dosing, and 4, 6, 12, 24, and 28 hours post dosing. Three rats are used for each time point, and the reported ganaxolone levels are the mean of ganaxolone plasma or brain levels of all three rats. Blood is collected by retro-orbital bleed or cardiac puncture. Blood samples are collected into K²⁺ EDTA coated tubes. Plasma samples are prepared by spinning blood in a refrigerated centrifuge (3000 rpm for 10 min at 4° C.

What is claimed is:

1. A sustained release injectable neurosteroid formulation comprising neurosteroid particles having a D50 of less than 10 microns, the neurosteroid particles comprising
a) a neurosteroid of Formula I: 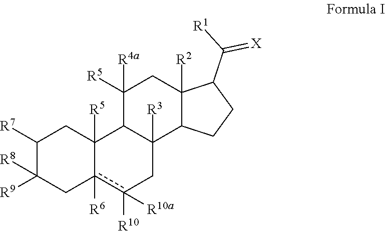
or a pharmaceutically acceptable salt thereof, wherein: [symbol]
is a double or single bond;
X is O, S, or NR¹¹;
R¹ is hydrogen, hydroxyl, optionally substituted alkyl, optionally substituted heteroalkyl, optionally substituted heteroaryl, optionally substituted heteroarylalkyl, optionally substituted aryl, or optionally substituted arylalkyl;
R⁴ is hydrogen, optionally substituted alkyl, optionally substituted cycloalkyl, or optionally substituted (cycloalkyl)alkyl, or —OR⁴⁰, where R⁴⁰ is hydrogen, optionally substituted alkyl, optionally substituted cycloalkyl, or optionally substituted (cycloalkyl)alkyl, or optionally substituted C₃-C₆carbocycle;
R^{4a} is hydrogen or R⁴ and R^{4a} are taken together to form an oxo (═O) group;
R², R³, R⁵, and R⁶, are each independently hydrogen, hydroxyl, halogen, optionally substituted alkyl, optionally substituted cycloalkyl, or optionally substituted (cycloalkyl)alkyl, or optionally substituted heteroalkyl;
R⁷ is hydrogen, halogen, optionally substituted alkyl, optionally substituted C₃-C₆carbocycle, optionally substituted (C₃-C₆carbocycle)alkyl or —OR⁷⁰ where R⁷⁰ is hydrogen, optionally substituted alkyl, optionally substituted C₃-C₆carbocycle, or optionally substituted (C₃-C₆carbocycle)alkyl;
R⁸ is hydrogen, optionally substituted alkyl or optionally substituted C₃-C₆carbocycle, and R⁹ is hydroxyl; or
R⁸ and R⁹ are taken together to form an oxo group;
R¹⁰ is hydrogen, halogen, hydroxyl, optionally substituted alkyl, optionally substituted heteroalkyl, optionally substituted C₃-C₆carbocyle, or optionally substituted (C₃-C₆carbocycle)alkyl, and R^{10a} is hydrogen, halogen, or optionally substituted alkyl, provided that if [symbol] is a double bond R^{10a} is absent;
each alkyl is a C₁-C₁₀alkyl and optionally contains one or more single bonds replaced by a double or triple bond;
each heteroalkyl group is an alkyl group in which one or more methyl group is replaced by an independently chosen —O—, —S—, —N(R¹¹)—, —S(═O)— or —S(═O)₂—, where R¹¹ is independently chosen at each occurrence and is hydrogen, alkyl, or alkyl in which one or more methylene group is replaced by —O—, —S—, —NH, or —N-alkyl; and
b) at least one polymeric surface stabilizer;
wherein the formulation provides an effective plasma concentration of the compound at steady state for at least 48 hours.

2. The sustained release injectable neurosteroid formulation of claim 1, wherein
R¹ is a group of the formula 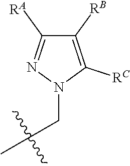
each instance of R^{A}, R^{B}, and R^{C} is, independently, hydrogen, halogen, —NO₂, —CN, —OR^{GA}, —N(R^{GA})₂, —C(═O)R^{GA}, —C(═O)OR^{GA}, —OC(═O)R^{GA}, —OC(═O)OR^{GA}, —C(═O)N(R^{GA})₂, —N(R^{GA})C(═O)R^{GA}, —OC(═O)N(R^{GA})₂, —N(R^{GA})C(═O)OR^{GA}, —N(R^{GA})C(═O)N(R^{GA})₂, —SR^{GA}, —S(O)R^{GA}, e.g., —S(═O)R^{GA}, —S(═O)₂R^{GA}, —S(═O)₂OR^{GA}, —OS(═O)₂R^{GA}, —S(═O)₂N(R^{GA})₂, —N(R^{GA})S(═O)₂R^{GA}, optionally substituted alkyl, optionally substituted C₃-C₆ carbocyclyl, or optionally substituted 3- to 6-membered heterocyclyl; and where instance of R^{GA} is independently hydrogen, optionally substituted alkyl, optionally substituted C₃-C₆ carbocyclyl, optionally substituted 3- to 6-membered heterocyclyl, optionally substituted aryl, optionally substituted heteroaryl, an oxygen protecting group when attached to oxygen, nitrogen protecting group when attached to nitrogen, or two R^{GA} groups are taken with the intervening atoms to form a substituted or unsubstituted heterocyclyl or heteroaryl ring.

3. The formulation of claim 1, wherein the neurosteroid is allopregnanolone, ganaxolone, alphaxalone, alphadolone, hydroxydione, minaxolone, pregnanolone, acebrochol, tetrahydrocorticosterone, isopregnanolone, or a compound of the formula 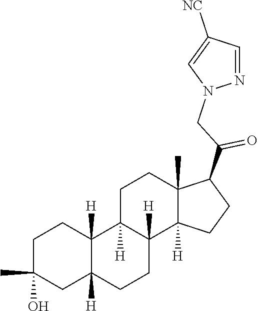
or a pharmaceutically acceptable salt of any of the foregoing.

4. The formulation of claim 3, wherein the polymeric surface stabilizer is hydroxyethyl starch, dextran, povidone, or a mixture of any the foregoing.

5. The formulation of claim 4, wherein the formulation comprises
an additional surface stabilizer and the additional surface stabilizer is an ionic or nonionic surfactant; and optionally,
an antifoaming agent.

6. The formulation of claim 5, wherein
the polymeric surface stabilizer is hydroxyethyl starch;
the surfactant is sodium cholate, sodium deoxycholate, sodium cholesterol sulfate, or a mixture of any of the foregoing; and.
the antifoaming agent is simethicone.

7. The formulation of claim 5 additionally comprising a cryoprotectant, wherein the cryoprotectant is sucrose, dextrose, lactose, D-sorbitol, mannitol, or a mixture of any of the foregoing.

8. The formulation of claim 5 additionally comprising one or more of the following
(a) 0.5% to 1.5% sodium chloride (weight percent);
(b) a buffer;
(c) a preservative, wherein the preservative is benzyl alcohol, chlorbutanol, 2-ethoxyethanol, parabens (including methyl, ethyl, propyl, butyl, and combinations), benzoic acid, sorbic acid, chlorhexidene, phenol, 3-cresol, thimerosal, a phenylmercurate salt, or a mixture of any of the foregoing.

9. The formulation of claim 1, wherein
the neurosteroid is ganaxolone or allopregnanolone;
the polymeric surface stabilizer is selected from hydroxyethyl starch, dextran, povidone, and a mixture of any of the foregoing, wherein the (wt:wt) ratio of the neurosteroid to the polymeric surface stabilizer is about 4:1 to about 0.5:1; and
the formulation comprises an additional surface stabilizer and the additional surface stabilizer is a surfactant, selected from sodium deoxycholate, sodium cholesterol sulfate, and a mixture of any of the foregoing; wherein the ratio of neurosteroid to surfactant (w:w) is about 10:1.5 to about 10:0.1.

10. The formulation of claim 9, wherein the formulation comprises neurosteroid particles have a D50 of 1 to 5 microns.

11. The formulation of claim 10, wherein upon injection of the formulation into a patient the formulation release neurosteroid over a period of at least 48 hours at least 4 weeks.

12. The formulation of any claim 10, wherein the formulation is an aqueous suspension comprising 1 mg/ml to 300 mg/ml ganaxolone.

13. The formulation of claim 1, wherein the formulation is an aqueous formulation comprising
(a) neurosteroid particles having a D50 of 1 to 5 microns, the neurosteroid particles comprising ganaxolone, wherein the weight percent of the ganaxolone is 5 to 20%;
(b) a polymeric surface stabilizer selected from hydroxy ethyl starch, dextran, and povidone, wherein the weight percent of the polymeric surface stabilizer is 2 to 50%;
(c) an additional surface stabilizer wherein the additional surface stabilizer is an ionic or nonionic surfactant selected from sodium cholate, sodium deoxycholate, and sodium cholesterol sulfate, wherein the weight percent of surfactant is 0.1% to 2.0%; and
(d) optionally, an antifoaming agent.

14. The formulation of claim 13, wherein
the polymeric surface stabilizer is hydroxyethyl starch 130/0.4 and the weight percent of the polymeric surface stabilizer is about 5% to about 20%; and
the additional surface stabilizer is sodium deoxycholate, wherein the weight percent of sodium deoxycholate is about 0.75%; and
simethicone, wherein the weight percent of simethicone is 0.009%.

15. A method of treating a patient having seizures, comprising administering the formulation of claim 13 to the patient.

16. A method of treating a patient having anxiety, agitation, depression, schizophrenia, post-traumatic stress disorder, or tremors, comprising administering the formulation of claim 13 to the patient.

17. The method of claim 16, wherein the depression is post-partum depression.

18. The method of claim 14, wherein the dosage of ganaxolone release from the formulation is from about 1 mg/kg/day to about 200 mg/kg/day.

19. The method of claim 14, wherein the formulation provides a ganaxolone plasma concentration at steady state of at least 10 ng/ml for a period of at least 1 week.

20. The method of claim 19, wherein the formulation provides a ganaxolone plasma concentration at steady state of 10 ng/ml to 800 ng/ml for at least 1 week.
